(19) United States
(12) Patent Application Publication
(10) Pub. No.: US 2006/0292073 A1
(43) Pub. Date: Dec. 28, 2006

(54) Stereoselective Synthesis of Amino Acid Analogs for Tumor Imaging

(75) Inventors: Mark M. Goodman, Atlanta, GA (US); Weiping Yu, Lilburn, GA (US)

Correspondence Address: GREENLEE WINNER AND SULLIVAN P C, 4875 PEARL EAST CIRCLE, SUITE 200, BOULDER, CO, 80301, US

(73) Assignee: Emory University, Atlanta, GA (US)

(21) Appl. No.: 11/425,051

(22) Filed: Jun. 19, 2006

Related U.S. Application Data
(60) Provisional application No. 60/693,385, filed on Jun. 23, 2005.

(51) Int. Cl. A61K 51/00; C07F 13/00; C07D 345/00; C07C 61/04

(52) U.S. Cl. 424/1.11; 540/1; 548/953; 549/510; 549/88; 562/505; 534/14

(57) ABSTRACT

The radiolabeled non-natural amino acid 1-amino-3-cyclobutane-1-carboxylic acid (ACBC) and its analogs are candidate tumor imaging agents useful for positron emission tomography and single photon emission computed tomography due to their selective affinity for tumor cells. The present invention provides methods for stereo-selective synthesis of syn-ACBC analogs. The disclosed synthetic strategy is reliable and efficient and can be used to synthesize a gram quantity of various syn-isomers of the ACBC analogs, particularly, syn-[¹⁸F]-1-amino-3-fluorocyclobutane-1-carboxylic acid (FACBC) and syn-[¹²³I]-1-amino-3-iodocyclobutane-1-carboxylic (IACBC) acid analogs.

### CROSS-REFERENCE TO RELATED APPLICATIONS

This application claims priority from U.S. Provisional Application No. 60/693,385, filed Jun. 23, 2005, which is incorporated herein in its entirety to the extent not inconsistent herewith.

### ACKNOWLEDGEMENT OF FEDERAL RESEARCH SUPPORT

This invention was made with government support under Grant No. 5-R21-CA-098891 awarded by the National Institutes of Health. The government has certain rights in this invention.

### BACKGROUND OF THE INVENTION

This invention relates to a method of synthesizing syn-amino acid analogs and compounds synthesized according to the merthod, particularly syn-1-amino-3-cyclobutane-1-carboxylic acid (ACBC) analogs. The amino acid analogs of the invention have specific binding in a biological system and capable of being used for positron emission tomography (PET) and single photon emission (SPECT) imaging methods.

The development of radiolabeled amino acids for use as metabolic tracers to image tumors using positron emission tomography (PET) and single photon emission computed tomography (SPECT) has been underway for some time. Although radiolabeled amino acids have been applied to a variety of tumor types, their application to intracranial tumors has received considerable attention due to potential advantages over other imaging modalities. After surgical resection and/or radiotherapy of brain tumors, conventional imaging methods such as CT and MRI do not reliably distinguish residual or recurring tumor from tissue injury due to the intervention and are not optimal for monitoring the effectiveness of treatment or detecting tumor recurrence [Buonocore, E (1992), Clinical Positron Emission Tomography. Mosby-Year Book, Inc. St. Louis, Mo., pp 17-22; Langleben, D D et al. (2000), J. Nucl. Med. 41:1861-1867].

The leading PET agent for diagnosis and imaging of neoplasms, 2-[¹⁸F]fluorodeoxyglucose (FDG), has limitations in the imaging of brain tumors. Normal brain cortical tissue shows high [¹⁸F]FDG uptake as does inflammatory tissue which can occur after radiation or surgical therapy; these factors can complicate the interpretation of images acquired with [¹⁸F]FDG [Griffeth, L K et al. (1993), Radiology. 186:3744; Conti, P S (1995)].

A number of reports indicate that PET and SPECT imaging with radiolabeled amino acids better define tumor boundaries within normal brain than CT or MRI, allowing better planning of treatment [Ogawa, T et al. (1993), Radiology. 186: 45-53; Jager, P L et al. (2001), Nucl. Med., 42:432-445]. Additionally, some studies suggest that the degree of amino acid uptake correlates with tumor grade, which could provide important prognostic information [Jager, P L et al. (2001) J. Nucl. Med. 42:432-445].

Amino acids are required nutrients for proliferating tumor cells. A variety of amino acids containing the positron emitting isotopes carbon-11 and fluorine-18 have been prepared. They have been evaluated for potential use in clinical oncology for tumor imaging in patients with brain and systemic tumors and may have superior characteristics relative to 2-[¹⁸F]FDG in certain cancers. These amino acid candidates can be subdivided into two major categories. The first category is represented by radiolabeled naturally occurring amino acids such as [¹¹C]valine, L-[¹¹C]leucine, L-[¹¹C]methionine (MET) and L-[1-¹¹C]tyrosine, and structurally similar analogues such as 2-[¹⁸F]fluoro-L-tyrosine and 4-[¹⁸F]fluoro-L-phenylalanine. The movement of these amino acids across tumor cell membranes predominantly occurs by carrier mediated transport by the sodium-independent leucine type “L” amino acid transport system. The increased uptake and prolonged retention of these naturally occurring radiolabeled amino acids into tumors in comparison to normal tissue is due in part to significant and rapid regional incorporation into proteins. Of these radiolabeled amino acids, [¹¹C]MET has been most extensively used clinically to detect tumors. Although [¹¹C]MET has been found useful in detecting brain and systemic tumors, it is susceptible to in vivo metabolism through multiple pathways, giving rise to numerous radiolabeled metabolites. Thus, graphical analysis with the necessary accuracy for reliable measurement of tumor metabolic activity is not possible. Studies of kinetic analysis of tumor uptake of [¹¹C]MET in humans strongly suggest that amino acid transport may provide a more sensitive measurement of tumor cell proliferation than protein synthesis.

The shortcomings associated with [¹¹C]MET may be overcome with a second category of amino acids. These are non-natural amino acids such as 1-aminocyclobutane-1-[¹¹C]carboxylic acid ([¹¹C]ACBC). The advantage of [¹¹C]ACBC in comparison to [¹¹C]MET is that it is not metabolized. A significant limitation in the application of carbon-11 amino acids for clinical use is the short 20-minute half-life of carbon-11. The 20-minute half-life requires an on-site particle accelerator for production of the carbon-11 amino acid. In addition only a single or relatively few doses can be generated from each batch production of the carbon-11 amino acid. Therefore carbon-11 amino acids are poor candidates for regional distribution for widespread clinical use.

In order to overcome the physical half-life limitation of carbon-11, we have recently focused on the development of several new fluorine-18 labeled non-natural amino acids, some of which have been disclosed in U.S. Pat. Nos. 5,808,146 and 5,817,776, both of which are incorporated herein by reference. These include anti-1-amino-3-[¹⁸F]fluorocyclobutyl-1-carboxylic acid (anti-[¹⁸F]FACBC), syn-1-amino-3-[¹⁸F]fluorocyclobutyl-1-carboxylic acid (syn-[¹⁸F]FACBC) syn- and anti-1-amino-3-[¹⁸F]fluoromethyl-cyclobutane-1-carboxylic acid (syn- and anti-[¹⁸F]FMACBC). These fluorine-18 amino acids can be used to image brain and systemic tumors in vivo based upon amino acid transport with the imaging technique Positron Emission Tomography (PET). Our development involved fluorine-18 labeled cyclobutyl amino acids that move across tumor capillaries by carrier-mediated transport involving primarily the “L” type large, neutral amino acid and to a lesser extent the “A” type amino acid transport systems. Our preliminary evaluation of cyclobutyl amino acids labeled with positron emitters, which are primarily substrates for the “L” transport system, has shown excellent potential in clinical oncology for tumor imaging in patients with brain and systemic tumors. The primary reasons for proposing ¹⁸F-labeling of cyclobutyl/branched amino acids instead of ¹¹C (t_{1/2}=20 min.) are the substantial logistical and economic benefits gained with using ¹⁸F instead of ¹¹C-labeled radiopharmaceuticals in clinical applications. The advantage of imaging tumors with ¹⁸F-labeled radiopharmaceuticals in a busy nuclear medicine department is primarily due to the longer half-life of ¹⁸F (t_{1/2}=110 min.). The longer half-life of ¹⁸F allows off-site distribution and multiple doses from a single production lot of radio tracer. In addition, these non-metabolized amino acids may also have wider application as imaging agents for certain systemic solid tumors that do not image well with 2-[¹⁸F]FDG PET. WO 03/093412, which is incorporated herein by reference, further discloses examples of fluorinated analogs of α-aminoisobutyric acid (AIB) such as 2-amino-3-fluoro-2-methylpropanoic acid (FAMP) and 3-fluoro-2-methyl-2-(methylamino)propanoic acid (N-MeFAMP) suitable for labeling with ¹⁸F and use in PET imaging. AIB is a nonmetabolizable α,α-dialkyl amino acid that is actively transported into cells primarily via the A-type amino acid transport system. System A amino acid transport is increased during cell growth and division and has also been shown to be upregulated in tumor cells [Palacin, M et al. (1998), Physiol. Rev. 78: 969-1054; Bussolati, O et al. (1996), FASEB J. 10:920-926]. Studies of experimentally induced tumors in animals and spontaneously occurring tumors in humans have shown increased uptake of radiolabeled AIB in the tumors relative to normal tissue [Conti, P S et al. (1986), Eur. J. Nucl. Med. 12:353-356; Uehara, H et al. (1997), J. Cereb. Blood Flow Metab. 17:1239-1253]. The N-methyl analog of AIB, N-MeAIB, shows even more selectivity for the A-type amino acid transport system than AIB [Shotwell, M A et al. (1983), Biochim. Biophys. Acta. 737:267-84]. N-MeAIB has been radiolabeled with carbon-11 and is metabolically stable in humans [Någren, K et al. (2000), J. Labelled Cpd. Radiopharm. 43:1013-1021].

Although the advantages of the amino acid analogs containing positron emitting isotopes for tumor imaging in patients with brain and systemic tumors have been well recognized in the art, there is still a need for a reliable and efficient synthetic method which can provide a large quantity of stereo-specific isomers of these compounds. As a candidate compound makes the transition from validation studies in cell and animal models to application in humans, the synthetic techniques employed must be adapted to allow routine, reliable production of the compound. Towards this end, the inventors herein developed a reliable stereoselective synthetic strategy for producing syn-1-amino-3-cyclobutane-1-carboxylic acid (ACBC) analogs. It will be apparent in the description below that this stereoselective synthetic strategy is applicable in synthesizing a variety of amino acid analogs, particularly those containing the radiotracers for tumor imaging with PET and SPECT.

### SUMMARY OF THE INVENTION

The invention provides a synthetic strategy which yields a specific stereo isomer of the key precursor for synthesizing an amino acid analog in syn isomeric form. This strategy is particularly useful in synthesizing syn-1-amino-3-cyclobutane-1-carboxylic acid (ACBC) analogs. The key step in the synthesis involves reduction of precursor synthons to the trans-alcohols which are converted to the final product in syn-isomeric form. The synthetic strategy disclosed herein is reliable, efficient and allows gram scale preparations of the key precursor for the radiosynthesis of syn-ACBC analogs. In addition, the synthetic strategy disclosed herein incorporates a suitable isotope as a last step to maximize the useful life of the isotope.

The present invention provides trans-alcohols having the formula:

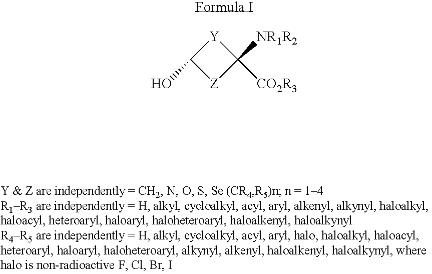

The invention also provides methods for synthesis of trans-alcohols having the general structure of formula 1. The key step in the synthesis of the trans-alcohols of the formula is a direct metal hydride reduction employing polymer bound reducing agents (e.g., Aldrich 32,864-2 Borohydride polymer supported on amberlite IRA 400; Aldrich 52,630-4 Cyanoborohydride polymer supported; Aldrich 35,994-7 Borohydride polymer supported on amberlite A-26; Aldrich 59,603-5 Zincborohydride polymer bound). Scheme 3 herein exemplifies this reaction using lithium triisobutylborane and ZnCl₂.

The synthetic strategy disclosed can be used to prepare syn-isomers of a variety of amino acid compounds for use in detecting and evaluating brain and body tumors and other uses. These compounds combine the advantageous properties of 1-amino-cycloalkyl-1-carboxylic acid, namely, their rapid uptake and prolonged retention in tumors with the properties of halogen substituents, including certain useful halogen isotopes including fluorine-18, iodine-123, iodine-125, iodine-131, bromine-75, bromine-76, bromine-77, bromine-82, astatine-210, astatine-211, and other astatine isotopes. In addition, the compounds can be labeled with technetium and rhenium isotopes using chelated complexes. See WO 03/093412 and U.S. Pat. No. 5,817,776 for detailed description.

The syn-amino acid analogs that can be made using the inventive synthetic strategy involving trans-alcohols include but are not limited to compounds having the following formula:

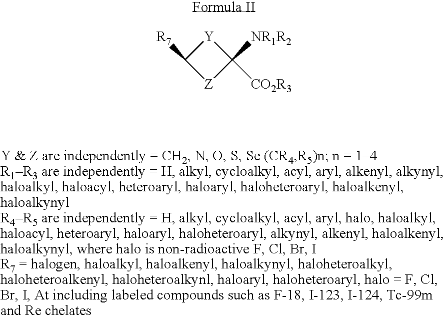

Specific radio-labeled amino acid analogs that can be made using the inventive methods disclosed herein include but are not limited to fluoro-, bromo- or iodo-substituted cyclopropyl, cyclobutyl, cyclopentyl, cyclohexyl, cyclcoheptyl, cyclcooctyl, cyclcononyl, cyclcodecyl amino acids having the structure shown above or alicyclic compounds containing a heteroatom, i.e. N, O and S and Se.

The amino acid compounds made according to the invention have a high specificity for tumor tissue when administered to a subject in vivo. Accordingly, the invention also provides pharmaceutical and diagnostic compositions comprising the syn-amino acid analogs made according to the inventive method. Preferred amino acid compounds show a target to non-target ratio of at least 2:1, are stable in vivo and substantially localized to target within 1 hour after administration. Examples of preferred amino acid compounds include syn-[¹⁸F]-1-amino-3-fluorocyclobutane-1-carboxylic acid (FACBC), syn-[¹²³I]-1-amino-3-iodocyclobutane-1-carboxylic acid (IACBC) and syn-[¹⁸F]-1-amino-3-fluoroalkyl-cyclobutane-1-carboxylic acid, for example, syn-[¹⁸F]-1-amino-3-fluoromethyl-cyclobutane-1-carboxylic acid (FMACBC).

The amino acid analogs of the invention are useful as an imaging agent for detecting and/or monitoring tumors in a subject. The amino acid analog imaging agent is administered in vivo and monitored using a means appropriate for the label. Preferred methods of detecting and/or monitoring an amino acid analog imaging agent in vivo include Positron Tomography (PET) and Single Photon Emission Computer Tomography (SPECT).

### BRIEF DESCRIPTION OF THE DRAWINGS

FIG. 1 shows the in vivo uptake of compounds in 9 L tumors. The results were expressed as percent uptake relative to control after 60 minutes of injection. See Example 2 for details.

FIG. 2 shows the in vivo uptake of compounds in contralateral normal brain at 60 minutes post-injection.

FIG. 3 shows the ratio of the in vivo uptake of compounds in tumor vs. normal cells at 60 minutes post-injection. The ratio was obtained from the percent values shown in FIGS. 1 and 2.

### DETAILED DESCRIPTION OF THE INVENTION

This invention relates to new methods for synthesizing syn-amino acid analogs useful for tumor imaging among other uses. The inventors herein developed a synthetic strategy which allows a stereo-selective synthesis of the key precursor in the trans isomeric form for the synthesis of syn-ACBC analogs. The ACBC analogs made by the inventive synthetic strategy are substantially pure in syn-isomeric form. The term, “substantially pure” as used herein means that the product is at least 60% pure in its isomeric form, preferably 70% pure, more preferably above 90% pure in syn-isomeric form. All intermediate values from 60% to 100% and all intermediate ranges therein are intended to be included whether or not they were individually listed.

In general the terms and phrases used herein have their art-recognized meaning, which can be found by reference to standard texts, journal references and contexts known to those skilled in the art. The following definitions are provided to clarify their specific use in the context of the invention.

The term “pharmaceutically acceptable salt” as used herein refers to those carboxylate salts or acid addition salts of the compounds of the present invention which are suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “pharmaceutically acceptable salt” refers to the relatively nontoxic, inorganic and organic acid addition salts of compounds of the present invention. Also included are those salts derived from non-toxic organic acids such as aliphatic mono and dicarboxylic acids, for example acetic acid, phenyl-substituted alkanoic acids, hydroxy alkanoic and alkanedioic acids, aromatic acids, and aliphatic and aromatic sulfonic acids. These salts can be prepared in situ during the final isolation and purification of the compounds or by separately reacting the purified compound in its free base form with a suitable organic or inorganic acid and isolating the salt thus formed. Further representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate and laurylsulphonate salts, propionate, pivalate, cyclamate, isethionate, and the like. These may include cations based on the alkali and alkaline earth metals, such as sodium, lithium, potassium, calcium, magnesium, and the like, as well as, nontoxic ammonium, quaternary ammonium and amine cations including, but not limited to ammonium, tetramethylammonium, tetraethylammonium, methylamine, dimethylamine, trimethylamine, triethylamine, ethylamine, and the like. See, for example, Berge S. M, et al., Pharmaceutical Salts, J. Pharm. Sci. 66:1-19 (1977) which is incorporated herein by reference.

Similarly, the term, “pharmaceutically acceptable carrier,” as used herein, is an organic or inorganic composition which serves as a carrier/stabilizer/diluent of the active ingredient of the present invention in a pharmaceutical or diagnostic composition. In certain cases, the pharmaceutically acceptable carriers are salts. Further examples of pharmaceutically acceptable carriers include but are not limited to water, phosphate-buffered saline, saline, pH controlling agents (e.g. acids, bases, buffers), stabilizers such as ascorbic acid, isotonizing agents (e.g. sodium chloride), aqueous solvents, a detergent (ionic and non-ionic) such as polysorbate or TWEEN 80.

The term “alkyl” as used herein by itself or as part of another group refers to a saturated hydrocarbon which may be linear, branched or cyclic of up to 10 carbons, preferably 6 carbons, more preferably 4 carbons, such as methyl, ethyl, propyl, isopropyl, butyl, t-butyl, and isobutyl. The alkyl groups of the invention include those optionally substituted where one or more carbon atoms in backbone can be replaced with a heteroatom, one or more hydrogen atoms can be replaced with halogen or —OH. The term “aryl” as employed herein by itself or as part of another group refers to monocyclic or bicyclic aromatic groups containing from 5 to 12 carbons in the ring portion, preferably 6-10 carbons in the ring portion, such as phenyl, naphthyl or tetrahydronaphthyl. The one or more rings of an aryl group can include fused rings. Aryl groups may be substituted with one or more alkyl groups which may be linear, branched or cyclic. Aryl groups may also be substituted at ring positions with substituents that do not significantly detrimentally affect the function of the compound or portion of the compound in which it is found. Substituted aryl groups also include those having heterocyclic aromatic rings in which one or more heteroatoms (e.g., N, O or S, optionally with hydrogens or substituents for proper valence) replace one or more carbons in the ring.

The term “alkoxy” is used herein to mean a straight or branched chain alkyl radical, as defined above, unless the chain length is limited thereto, bonded to an oxygen atom, including, but not limited to, methoxy, ethoxy, n-propoxy, isopropoxy, and the like. Preferably the alkoxy chain is 1 to 6 carbon atoms in length, more preferably 1-4 carbon atoms in length.

“Acyl” group is a group which includes a —CO— group.

The term “monoalkylamine” as used herein by itself or as part of another group refers to an amino group which is substituted with one alkyl group as defined above.

The term “dialkylamine” as employed herein by itself or as part of another group refers to an amino group which is substituted with two alkyl groups as defined above.

The term “halo” employed herein by itself or as part of another group refers to chlorine, bromine, fluorine or iodine.

The term “heterocycle” or “heterocyclic ring”, as used herein except where noted, represents a stable 5- to 7-membered mono-heterocyclic ring system which may be saturated or unsaturated, and which consists of carbon atoms and from one to three heteroatoms selected from the group consisting of N, O, and S, and wherein the nitrogen and sulfur heteroatom may optionally be oxidized. Especially useful are rings contain one nitrogen combined with one oxygen or sulfur, or two nitrogen heteroatoms. Examples of such heterocyclic groups include piperidinyl, pyrrolyl, pyrrolidinyl, imidazolyl, imidazlinyl, imidazolidinyl, pyridyl, pyrazinyl, pyrimidinyl, oxazolyl, oxazolidinyl, isoxazolyl, isoxazolidinyl, thiazolyl, thiazolidinyl, isothiazolyl, homopiperidinyl, homopiperazinyl, pyridazinyl, pyrazolyl, and pyrazolidinyl, most preferably thiamorpholinyl, piperazinyl, and morpholinyl. sulfur atom (“S”) or a nitrogen atom (“N”). It will be recognized that when the heteroatom is nitrogen, it may form an NR^{a}R^{b} moiety, wherein R^{a} and R^{b} are, independently from one another, hydrogen or C₁₋₄ alkyl, C₂₋₄ aminoalkyl, C₁₋₄ haloalkyl, halobenzyl, or R^{a} and R^{b} are taken together to form a 5- to 7-member heterocyclic ring optionally having O, S or NR^{c} in said ring, where R^{c} is hydrogen or C₁₋₄ alkyl.

The compounds of the invention are useful as tumor binding agents and as NMDA receptor-binding ligands, and in radio-isotopic form are especially useful as tracer compounds for tumor imaging techniques, including PET and SPECT imaging. Particularly useful as an imaging agent are those compounds labeled with F-18 since F-18 has a half-life of 110 minutes, which allows sufficient time for incorporation into a radio-labeled tracer, for purification and for administration into a human or animal subject. In addition, facilities more remote from a cyclotron, up to about a 200 mile radius, can make use of F-18 labeled compounds.

SPECT imaging employs isotope tracers that emit high energy photons (γ-emitters). The range of useful isotopes is greater than for PET, but SPECT provides lower three-dimensional resolution. Nevertheless, SPECT is widely used to obtain clinically significant information about analog binding, localization and clearance rates. A useful isotope for SPECT imaging is [¹²³I], a -γ-emitter with a 13.3 hour half life. Compounds labeled with [¹²³I] can be shipped up to about 1000 miles from the manufacturing site, or the isotope itself can be transported for on-site synthesis. Eighty-five percent of the isotope's emissions are 159 KeV photons, which is readily measured by SPECT instrumentation currently in use.

Accordingly, the compounds of the invention can be rapidly and efficiently labeled with [¹²³I] for use in SPECT analysis as an alternative to PET imaging. Furthermore, because of the fact that the same compound can be labeled with either isotope, it is possible to compare the results obtained by PET and SPECT using the same tracer.

Other halogen isotopes can serve for PET or SPECT imaging, or for conventional tracer labeling. These include ⁷⁵Br, ⁷⁶Br, ⁷⁷Br and ⁸²Br as having usable half-lives and emission characteristics. In general, the chemical means exist to substitute any halogen moiety for the described isotopes. Therefore, the biochemical or physiological activities of any halogenated homolog of the compounds of the invention are now available for use by those skilled in the art, including stable isotope halogen homologs. Astatine can be substituted for other halogen isotopes, [²¹⁰At] emits alpha particles with a half-life of 8.3 h. At-substituted compounds are therefore useful for tumor therapy, where binding is sufficiently tumor-specific.

The invention provides methods for tumor imaging using PET and SPECT. The methods entail administering to a subject (which can be human or animal, for experimental and/or diagnostic purposes) an image-generating amount of a compound of the invention, labeled with the appropriate isotope and then measuring the distribution of the compound by PET if [¹⁸F] or other positron emitter is employed, or SPECT if [¹²³I] or other gamma emitter is employed. An image-generating amount is that amount which is at least able to provide an image in a PET or SPECT scanner, taking into account the scanner's detection sensitivity and noise level, the age of the isotope, the body size of the subject and route of administration, all such variables being exemplary of those known and accounted for by calculations and measurements known to those skilled in the art without resort to undue experimentation.

It will be understood that compounds of the invention can be labeled with an isotope of any atom or combination of atoms in the structure. While [¹⁸F], [¹²³I] and [¹²⁵I] have been emphasized herein as being particularly useful for PET, SPECT and tracer analysis, other uses are contemplated including those flowing from physiological or pharmacological properties of stable isotope homologs and will be apparent to those skilled in the art.

The compounds of the invention can also be labeled with technetium (Tc) via Tc adducts. Isotopes of Tc, notably Tc^{99m}, have been used for tumor imaging. The present invention provides Tc-complexed adducts of compounds of the invention, which are useful for tumor imaging. The adducts are Tc-coordination complexes joined to the cyclic amino acid by a 4-6 carbon chain which can be saturated or possess a double or triple bond. Where a double bond is present, either E (trans) or Z (cis) isomers can be synthesized, and either isomer can be employed. The inventive compounds labeled with Tc are synthesized by incorporating the ^{99m}Tc isotope as a last step to maximize the useful life of the isotope.

U.S. Pat. No. 5,817,776 discloses a ten step reaction sequence for the synthesis of (anti-[¹⁸F]-1-amino-3-fluorocyclobutane-1-carboxylic acid (FACBC)) which involved a labor-intensive semi-preparative high pressure liquid chromatography separation following step 4 of a 75:25 mixture of the key intermediates, cis 1-amino-3-benzyloxycyclobutane-1-carboxylic acid and trans 1-amino-3-benzyloxycyclobutane-1-carboxylic acid, respectively. The purified major isomer, cis 1-amino-3-benzyloxycyclobutane-1-carboxylic acid, was then converted to the triflate precursor in a six-step reaction sequence.

In an effort to improve the synthetic methods, the inventors developed the stereo-selective synthesis of trans-(anti-) 1-amino-3-[¹⁸F]fluorocyclobutane-1-carboxylic acid (anti-[¹⁸F]FACBC) to large scale syntheses of both the precursor for radiolabeling, cis 1-t-butyl carbamate-3-trifluoromethane sulfonoxy-1-cyclobutane-1-carboxylic methyl ester (8), and trans 1-amino-3-fluorocyclobutane-1-carboxylic acid (anti-FACBC) (10). Schemes 1 and 2 illustrate the steps of synthesizing anti-FACBC. Using the synthetic steps shown, we were able to prepare the triflate precursor (8) from a seven-step reaction sequence. The key step in the synthesis is the preparation of the synthon 3-benzyloxy-cyclobutanone (2). Preparation of cyclobutanone 3 involved cyclization by treatment of 1-bromo-2-benzyloxy-3-bromopropane (1) with methylethyl-sulfoxide and n-butyl lithium. The ketone 2 was converted directly to the hydantoins 3 and 4 under Bucherer Strecker conditions. The 80:20 mixture of cis:trans hydantoins was easily purified by flash chromatography to give the desired cis hydantoin 4. The conversion of 4 to the triflate precursor, cis 1-t-butyl carbamate-3-trifluoromethane sulfonoxy-1-cyclobutane-1-carboxylic methyl ester (8) was carried out by the sequence of reactions described in U.S. Pat. No. 5,817,776. Utilizing this method we were able to prepare gram quantities of compound 9. [McConathy et al. (2003) Jour. of Applied Radiation and Isotopes, 58: 657-666].

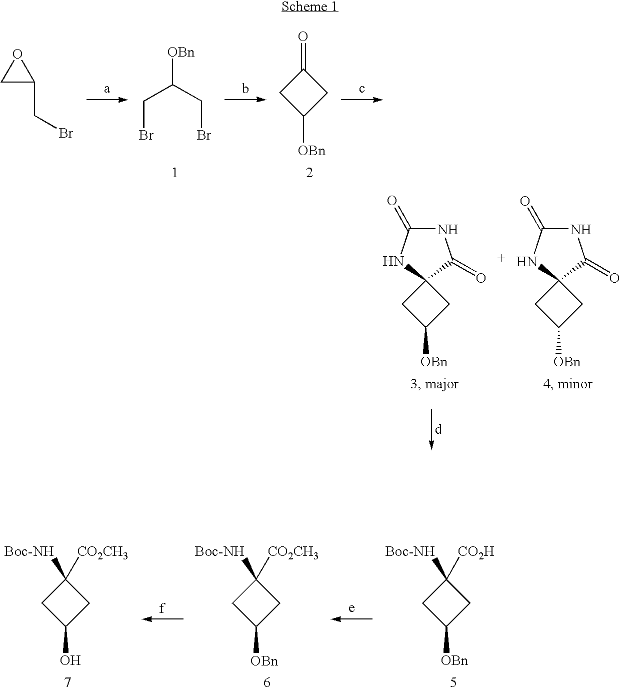

a) benzyl bromide, Hg₂Cl₂, 150° C.; b) nBuLi, CH₃S(O)CH₂SCH₃, THF then 35% HClO₄/Et₂O; c) NH₄(CO₃)₂, NH₄Cl, KCN, 1:1 EtOH:H₂O, 60° C. d) 3N NaOH, 180° C. then Boc₂O, 9:1 CH₃OH:Et₃N; e) (CH₃)₃SiCHN₂, 1:1 CH₃OH:THF; f) 10% Pd/C, H₂, CH₃OH.

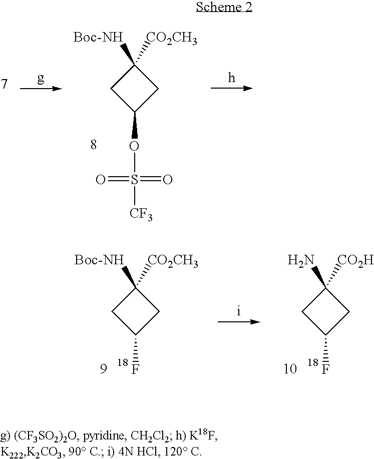

In order to obtain sufficient quantities of the amino acid analogs in syn-isomeric form for tumor imaging, in particular, cis-(syn-)1-amino-3-fluorocyclobutane-1-carboxylic acid (syn-FACBC), a new general synthetic approach was developed as shown in Schemes 3-5, for a large scale production of trans-1-t-butyl carbamate-3-trifluoromethane sulfonoxy-1-cyclobutane-1-carboxylic methyl ester. The key step in the syntheses involved reduction of the synthons 1-trifuoroacetamide-cyclobutan-3-one-1-carboxylic methyl ester (11a), 1-phtalamide-cyclobutan-3-one-1-carboxylic methyl ester (11b), 1-t-butyl carbamate-cyclobutan-3-one-1-carboxylic methyl ester (11c) and 1-benzamide-cyclobutan-3-one-1-carboxylic methyl ester (11d). The ketones 11a-d were converted directly to the trans-(anti-) alcohols in 63-80% yield by treatment with lithium triisobutylborane and ZnCl₂. The method afforded 95:5, 97:3, 70:30 and 90:10 mixtures of trans:cis alcohols 12a, 12b, 12c and 12d, respectively.12a-12d were easily purified by flash chromatography to give the desired trans alcohols 12a-d. The conversion of 12a-d to the triflate precursors can be carried out by the sequence of reactions described in U.S. Pat. No. 5,817,776. The development of these synthetic approaches are essential to establish a readily available supply of the precursor for distribution to PET centers for future multicenter clinical trials to validate syn- and anti-FACBC as a valuable imaging agent for the diagnosis and management of treatment of cancer.

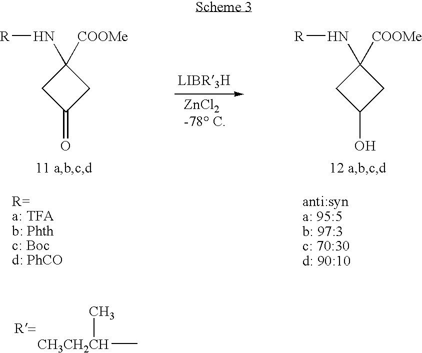

The above reaction was carried out in the following manner; to the solution of the ketone (11a, b, c, or d) in THF (anh.) was added 2 equivalent of ZnCl₂ (anh., in THF) at room temperature (rt) under Argon. The solution was stirred at room temperature for 30 min followed by the addition of 1.5 equivalent of LiBR′₃H at −78° C. The mixture was stirred at −78° C. for 2 hrs then at rt overnight. NH₄Cl (1N aq., 3 equivalent) was added and the mixture was stirred at rt for 30 min. The reaction was washed with brine, and aqueous phase was re-extracted with ethyl acetate. The combined organic phases were dried over sodium sulfate and concentrated to dryness. The product was purified on silica gel using 1:1 hexane and ehtyl acetate as eluant. The yields were approximately 63-80%.

Although the recation step shown in Scheme 3 specifically exemplifies the reduction of four synthons (11a-11d) to four trans-alcohols, 12a-12d, this stereo-selective synthetic step can be applied to the synthesis of a variety of trans-alcohols for syntheis of syn-amino acid analogs useful for tumor imaging. Scheme 4 below illustrates this aspect of the invention. 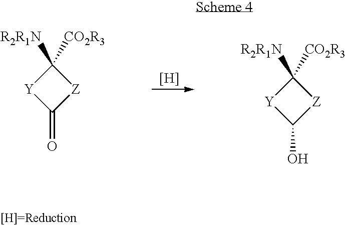
Scheme 5 exemplifies the steps for synthesis of syn-FACBC. 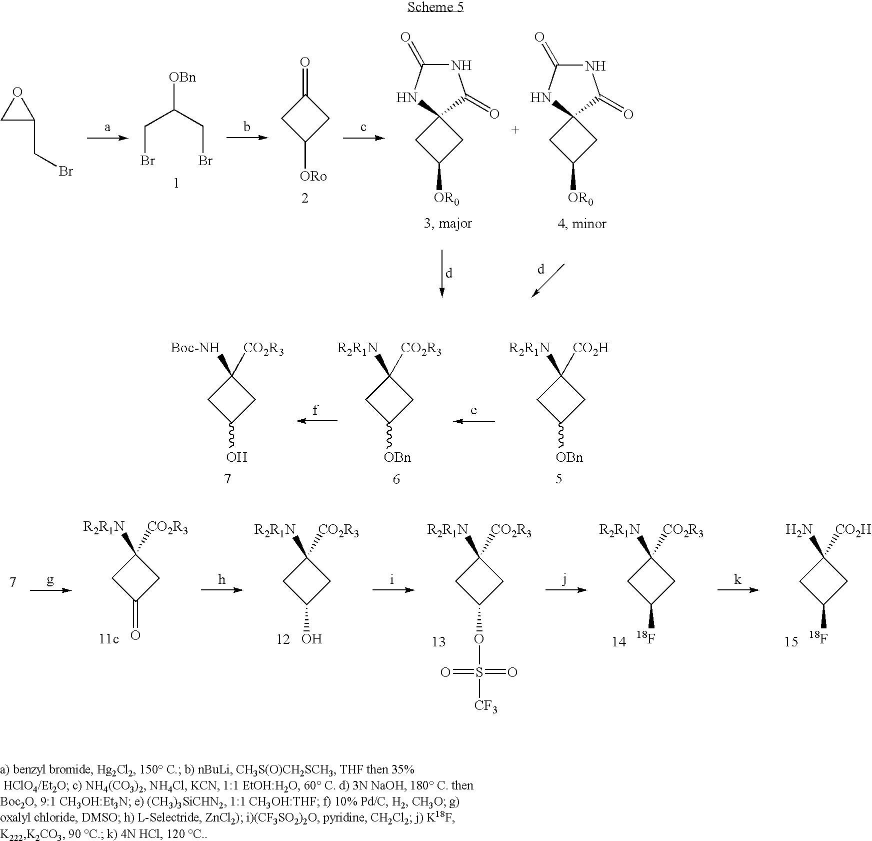
Scheme 6 exemplifies the synthesis of an amino acid analog, [¹⁸F]-1-amino4-fluoro-cyclohexane-1-carboxylic acid (FACHC) which can be synthesized using the stereo selective synthetic method disclosed herein. 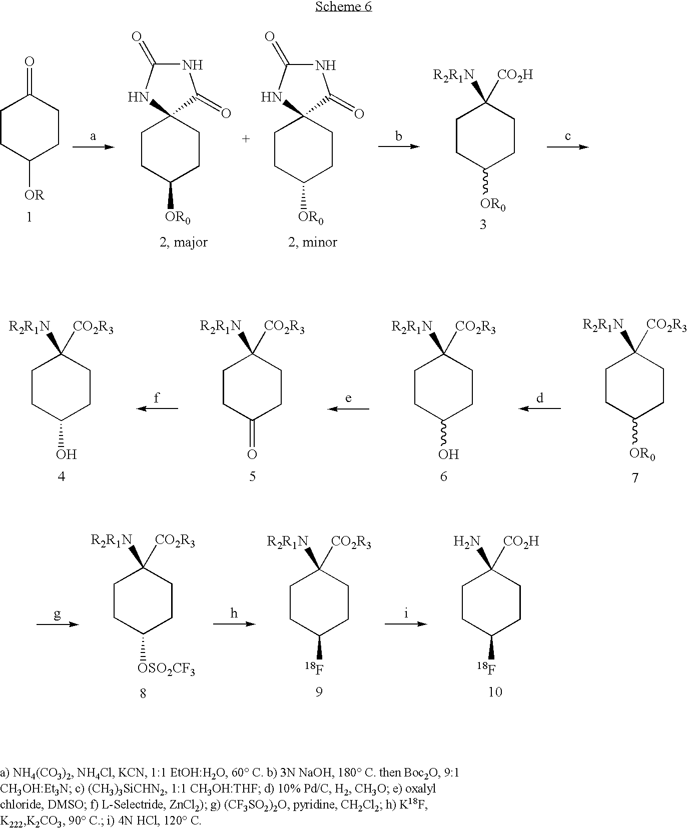
Scheme 7 shows the synthesis of syn/anti-1-amino-3-benzyloxycyclobutane-1-carboxylic acids 20 which is a key synthon used in the stereoselective synthetic method disclosed herein. 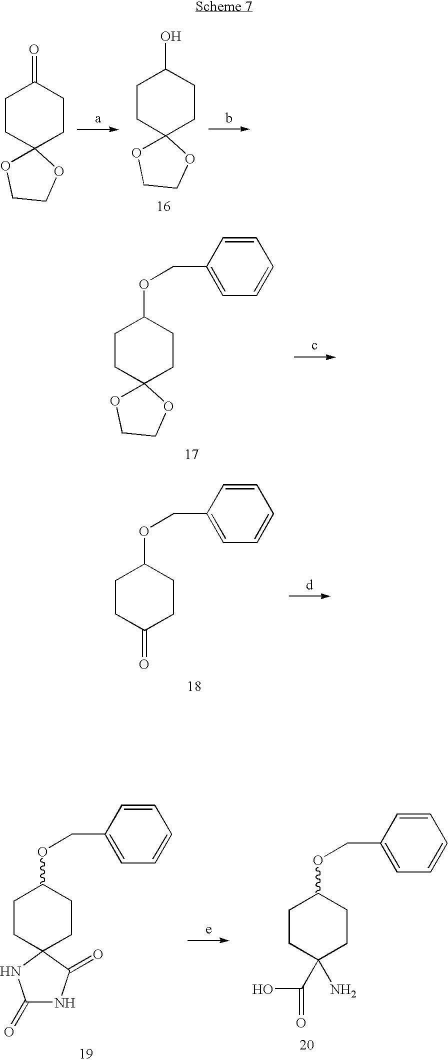
Scheme 8 shows the syntheses of 1-[N-(t-Butoxycarbonyl)amino]-4-cyclohexanon-1-carboxylic acid methyl ester (24), 1-Amino-4-cyclohexanon-1-carboxylic acid methyl ester (25), which are key cyclohexanone intermediates used in the stereoselective synthetic method disclosed herein. 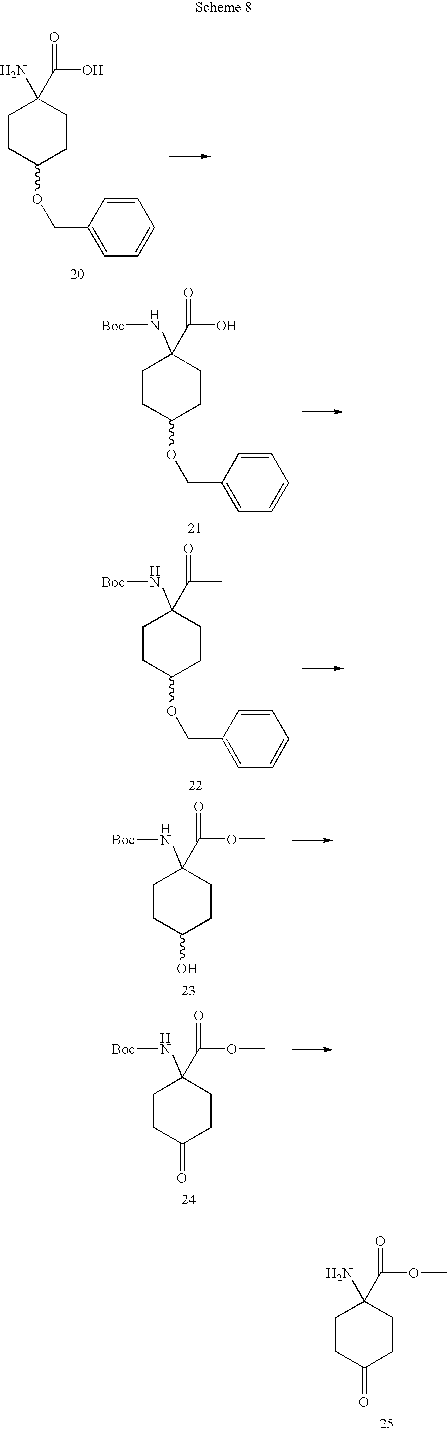
Scheme 9 shows the syntheses of syn/anti-1-[N-substituted-4-hydroxycyclohexane-1-carboxylic acid methyl esters 27a-d prepared in the stereoselective synthetic method disclosed herein.

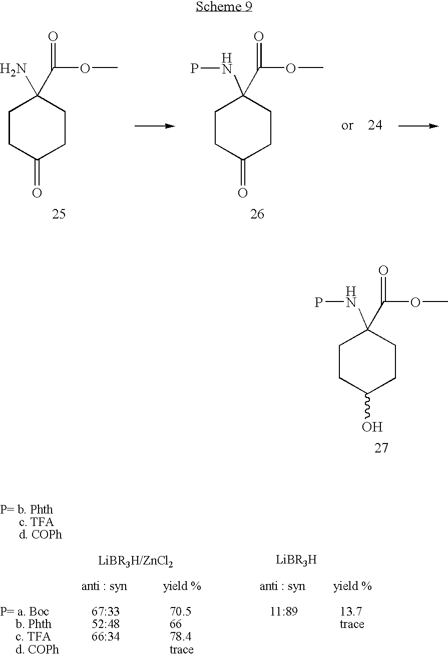

### EXAMPLES

The following descriptions provide exemplary syntheses of preferred embodiments of the present invention. However, one of ordinary skill in the art will appreciate that starting materials, reagents, solvents, temperature, solid substrates, synthetic methods, purification methods, analytical methods, and other reaction conditions other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### Example 1

### Synthesis of syn- and anti-[¹⁸F]1-amino-3-fluorocyclobutane-1-carboxylic acid (FACBC) (Schemes 1, 2 and 5)

The following methods were employed in procedures reported herein. [¹⁸F]-Fluoride was produced from a Seimens cyclotron using the ¹⁸O(p,n)¹⁸F reaction with 11 MeV protons on 95% enriched [¹⁸O] water. All solvents and chemicals were analytical grade and were used without further purification. Melting points of compounds were determined in capillary tubes by using a Buchi SP apparatus. Thin-layer chromatographic analysis (TLC) was performed by using 250-mm thick layers of silica gel G PF-254 coated on aluminum (obtained from Analtech, Inc. Newark, Del.). Column chromatography was performed by using 60-200 mesh silica gel (Sigma-Aldrich, St. Louis, Mo.). Infrared spectra (IR) were recorded on a Beckman 18A spectrophotometer with NaCl plates. Proton nuclear magnetic resonance spectra (¹H NMR) were obtained at 300 MHz with a Nicolet high-resolution instrument.

Synthesis of 1-bromo-2-benzyloxy-3-bromopropane 1:

In a flask fitted with a condenser, a mixture consisting of benzyl bromide (83 mL, 0.70 mol), epibromohydrin (60 mL, 0.70 mol) and mercury (I) chloride (120 mg, 0.25 mmol) was heated with stirring at 150° C. overnight. The product was isolated via vacuum distillation through a 30 cm Vigreux condenser (110-115° C., 0.5 mm Hg) to provide 1 (152 g, 70%) as a colorless liquid: ¹H NMR (CDCl₃) δ3.45 (4H, d, J=5.2), 3.66-3.71 (1H, m) 4.55 (2H, s) 7.19-7.27 (5H, m).

Synthesis of 3-benzyloxy cyclobutanone 2:

The preparation of the cyclobutanone 2 was based on the procedure reported by Ogura et al. (1984) Bull. Chem. Soc. Jpn. 57; 1637-42. A 2.4 eq portion of n-butyl lithium (1.6 M in hexane, 243 mL) was added dropwise to a solution containing 2.4 eq of methyl methylsulfinyl methylsulfide (41 mL, 0.39 mmoles) in 400 mL of tetrahydrofuran at −10° C. The reaction mix was then stirred at −10° C. for 2 hours and then cooled to −70° C. The yellow reaction mix was maintained at −70° C. and 1 equivalent of the dibromo species 1 (50 g, 0.16 mmoles) in 85 mL of tetrahydrofuran was added dropwise. The reaction mix was allowed to warm to room temperature overnight. The reaction mix was added to brine and extracted twice with ethyl acetate. The combined organic layers were subject to the usual work up to provide ˜60 mL of dark red-brown liquid. This mixture of syn- and anti-dithioketal S-oxide intermediates was purified in three portions via silica gel column chromatography (90 g silica). Less polar impurities were eluted first with 3:7 ethyl acetate:hexane followed by elution of product with pure ethyl acetate. A total of 23.8 grams of intermediate was obtained in this manner. In a second synthesis of 2 using identical conditions, 24.6 grams were obtained.

The syn- and anti-dithioketal S-oxide intermediates (48.4 g, 0.18 moles) were dissolved in 1200 mL of diethyl ether and treated with 68 mL of 35% perchloric acid. After overnight stirring, the reaction mix was neutralized with sodium bicarbonate followed by usual work up. Purification via silica gel column chromatography (15:85 ethyl acetate:hexane) provided the ketone 2 (23.6 g, 41% from 1) as an orange-yellow liquid: ¹H NMR δ3.11-3.29 (4H, m) 4.35-4.42 (1H, m) 4.53 (2H, s) 7.30-7.40 (5H, m).

Synthesis of cis/trans 5-(3-benzyloxycyclobutane)hydantoin 3:

To a solution of 10 eq of ammonium carbonate (125 g, 1.3 mol) and 4 eq of ammonium chloride (27.8 g, 0.52 mol) in 900 mL of water was added 1 eq of the cyclobutanone 2 (23.6 g, 0.13 mole) in 900 mL of ethanol. After stirring at room temperature for 30 minutes, a 4.5 eq portion of potassium cyanide (38 g, 0.58 mole) was added, and the reaction mix was heated at 60° C. overnight. The solvent was removed under reduced pressure, and the crude yellow solid was rinsed thoroughly with approximately 1 liter of water to remove salts. The white crystalline product (16.4 g, 51%) was obtained as a 5:1 mixture of syn:anti isomers. The isolated major isomer was obtained via silica gel column chromatography (2:98 methanol:dichloromethane). Using this procedure, purification of 1.0 g of the mixture on 95 g of silica gel provided 500-600 mg of pure 3 in a single run. syn-5-(3-benzyloxycyclobutane)hydantoin (3): ¹H NMR (CDCl₃) δ2.30-2.35 (2H, m) 2.87-2.92 (2H, m) 4.18-4.25 (1H, m) 4.46 (2H, s) 5.66 (1 H, broad s) 7.28-7.38 (5H, m) 7.55 (1H, broad s). anti-5-(3-benzyloxycyclobutane)hydantoin (4): ¹H NMR (CDCl₃) δ2.44-2.50 (2H, m) 2.77-2.83 (2H, m) 4.21-4.27 (1H, m) 4.46 (2H, s) 5.82 (1H, broad s) 7.29-7.38 (6H, m).

Synthesis of syn/anti-1-(N-(tert-butoxycarbonyl)amino)-3-benzyloxycyclobutane-1-carboxylic acid 5:

A suspension of compound 3 (1.35 g, 5.5 mmoles) in 30 mL of 3N sodium hydroxide was heated at 180° C. overnight in a sealed stainless steel vessel. After cooling, the reaction mix was neutralized to pH 6-7 with concentrated hydrochloric acid. After evaporation of water under reduced pressure, the resulting solid was extracted with 4×30 mL of hot ethanol. The combined ethanol extracts were concentrated, and the residue was dissolved in 50 mL of 9:1 methanol:triethylamine. To the solution was added a 1.3 eq portion of di-tert-butyl dicarbonate (1.56 g), and the solution was stirred at room temperature overnight. The solvent was removed under reduced pressure, and the crude product was stirred in a mixture of ice-cold 80 mL of ethyl acetate and ice-cold 80 mL of 0.2N hydrochloric acid for five minutes. The organic layer was retained, and the aqueous phase was extracted with 2×80 mL of ice-cold ethyl acetate. The combined organic layers were washed with 3×60 mL of water followed by usual work up. The N-Boc acid 5 (1.27 g, 72%) was obtained as a white solid suitable for use in the next step without further purification. ¹H NMR (CDCl₃) δ1.44 (9H, s) 2.21-2.26 (2H, m) 3.02-3.08 (2H, broad m) 4.12-4.19 (1H, m) 4.44 (2H, s) 5.18 (1H, broad s) 7.27-7.37 (5H, m).

Synthesis of syn/anti-1-(N-(tert-butoxycarbonyl)amino)-3-benzyloxycyclobutane-1-carboxylic acid methyl ester 6:

A 1.5 eq portion of 2.0 M trimethylsilyl diazomethane in hexane (1.4 mL) was added dropwise to a solution of the N-Boc acid 5 (600 mg, 1.87 mmoles) in 10 mL of 1:1 methanol:tetrahydrofuran. During the exothermic addition, significant gas evolution occurred. After 20 minutes of stirring, the reaction mix was concentrated under reduced pressure, and the crude product was purified via silica gel column chromatography (2:8 ethyl acetate:hexane). The N-Boc methyl ester 6 (0.45 g, 72%) was obtained as a white crystalline solid. ¹H NMR (CDCl₃) δ1.42 (9H, s) 2.24-2.36 (2H, broad m) 2.88-2.96 (2H, m) 3.75 (3H, s) 4.16-4.23 (1H, m) 4.44 (2H, s) 5.13 (1H, s) 7.27-7.36 (5H, m).

Synthesis of syn/anti-1-(N-(tert-butoxycarbonyl)amino)-3-hydroxycyclobutane-1-carboxylic acid methyl ester 7:

To a solution of 6 (450 mg, 1.34 mmoles) in 10 mL of CH₃OH under an argon atmosphere was added 200 mg of 10% Pd/C. The reaction mix was stirred overnight at room temperature under a hydrogen atmosphere. The suspension was then filtered over Celite® and concentrated under reduced pressure. Purificatioin via silica gel column chromatography (6:4 ethyl acetate:hexane) provided the alcohol 7 (200 mg, 61%) as a white crystalline solid: 134-135° C. (128-130° C. reported by Shoup and Goodman, J Labelled Compd Radiopharm, 1999; 42: 215-225. ¹H NMR (CDCl₃) δ1.45 (9H, s) 2.54-2.61 (2H, broad m) 2.98-3.04 (2H, m) 3.79 (3H, s) 4.26-4.34 (1H, broad m) 5.63 (1H, broad s). Anal. (C₁₁H₁₉NO₅) calculated C, 53.87; H, 7.81; N, 5.71; found C, 53.93; H, 8.00; N, 5.71.

Synthesis of compound 1-[N-(tert-butoxycarbonyl)amino]-cyclobutan-3-one-1-carboxylic acid methyl ester 11c.

To a 1.1 eq portion of oxalyl chloride (1.05 mL of 2M solution in dichloromethane) in 4 mL of dichloromethane at −50 to −60° C. under argon was added in a dropwise fashion 2.2 eq of dimethyl sulfoxide (290 μL) in 1 mL of dichloromethane. This solution was stirred for 3 minutes followed by the dropwise addition of isomerically impure 7 (458 mg, 1.9 mmole) dissolved in 2 mL dichloromethane and 0.8 mL of dimethyl sulfoxide. The reaction mix was stirred at −50 to −60° C. for 20 minutes, and then 5 eq of triethylamine (1.3 mL) was added. The reaction mix was stirred for 5 minutes, the cooling bath was removed, and the solution was stirred for an additional 15 minutes. The crude product was purified via silica gel column chromatography (1:4 ethyl acetate:hexane) to provide 11c (456 mg, 100% yield) as a white solid: 118-119° C. (ethyl acetate/hexane): ¹H NMR (CDCl₃) δ1.46 (9H, s) 3.49-3.66 (4H, m) 3.83 (3H, s) 5.47 (1H, broad s). Anal. (C₁₁H₁₇NO₅) calculated C, 54.31; H, 7.04; N, 5.76; found C, 54.50; H, 6.96; N, 5.61.

Synthesis of anti-1-(N-(tert-butoxycarbonyl)amino)-3-hydroxycyclobutane-1-carboxylic acid methyl ester 12c.

To the solution of the ketone (11c, 16.4 mg, 0.067 mmol) in 1 ml THF (anh.) was added ZnCl₂ (18 mg, 0.134 mmol, in THF) at rt under Ar. The solution was stirred at rt for 30 min followed by the addition of L-Selectride (19 mg, 0.10 mmol, in THF) at −78° C. The mixture was stirred at −78° C. for 2 hrs then at rt overnight. NH₄Cl (1N aq., 3 equivalent) was added and the mixture was stirred at rt for 30 min. The reaction was washed with brine, and aqueous phase was re-extracted with ethyl acetate. The combined organic phases were dried over sodium sulfate and concentrated to dryness. The product was purified on silica gel using 1:1 hexane and ethyl acetate as eluant. The product (12c, 16 mg, 100%) was a white solid: ¹H NMR (CDCl₃) δ1.44 (9H, s) 2.53-2.63 (4H, broad m) 3.77 (3H, s) 4.43-4.50 (1H, broad m) 5.02 (1H, broad s).

Synthesis of anti-1-(N-(tert-butoxycarbonyl)amino)-3-trifluoromethylsulfonoxycyclobutane-1-carboxylic acid methyl ester 13.

The alcohol 9 (10 mg, 0.04 mmoles) was dissolved in 2 mL of dichloromethane under an argon atmosphere. With ice-bath cooling, a 100 μL portion of pyridine was added followed by 4.5 eq portion of trifluoromethanesulfonic anhydride (30 μL). After stirring for 15 minutes, the solvent was removed under reduced pressure at room temperature. The crude product was purified via silica gel column chromatography (3:7 ethyl acetate:hexane) to provide the labeling precursor 13.

Synthesis of syn-[¹⁸F]1-amino-3-fluorocyclobutane-1-carboxylic acid (FACBC) 15:

[¹⁸F]-Fluoride was produced using the ¹⁸O (p,n)¹⁸F reaction with 11 MeV protons on 95% enriched [¹⁸O] water. After evaporation of the water and drying of the fluoride by acetonitrile evaporation, the protected amino acid triflate 13 (20 mg) was introduced in an acetonitrile solution (1 mL). The no carrier added (NCA) fluorination reaction was performed at 85° C. for 5 min in a sealed vessel in the presence of potassium carbonate and Kryptofix (Trademark Aldrich Chemical Co., Milwaukee, Wis.). Unreacted ¹⁸F was removed by diluting the reacting mixture with methylene chloride followed by passage through a silica gel Seppak which gave the ¹⁸F labeled product 14. Deprotection of 14 was achieved by using 1 mL of 6 N HCl at 115° C. for 15 min and then the aqueous solution containing syn-[¹⁸F]FACBC 15 was passed through an ion-retardation resin (AG 11A8 50-100 mesh).

Synthesis of anti-[¹⁸F]FACBC 10:

[¹⁸F]-Fluoride was produced using the ¹⁸O (p,n)¹⁸F reaction with 11 MeV protons on 95% enriched [¹⁸O] water. After evaporation of the water and drying of the fluoride by acetonitrile evaporation, the protected amino acid triflate syn-1-(N-(tert-butoxycarbonyl)amino)-3-trifluoromethanesulfonoxycyclobutane-1-carboxylic acid methyl ester (20 mg) was introduced in an acetonitrile solution (1 mL). The no carrier added (NCA) fluorination reaction was performed at 85° C. for 5 min in a sealed vessel in the presence of potassium carbonate and Kryptofix (Trademark Aldrich Chemical Co., Milwaukee, Wis.). Unreacted ¹⁸F was removed by diluting the reacting mixture with methylene chloride followed by passage through a silica gel Seppak which gave the ¹⁸F labeled product syn-1-(N-(tert-butoxycarbonyl)amino)-3-[¹⁸F]fluorocyclobutane-1-carboxylic acid methyl ester in 42% E.O.B. yield. Deprotection of syn-1-(N-(tert-butoxycarbonyl)amino)-3-[¹⁸F]fluorocyclobutane-1-carboxylic acid methyl ester was achieved by using 1 mL of 4 N HCl at 115° C. for 15 min and then the aqueous solution containing ¹⁸FACBC 13 was passed through an ion-retardation resin (AG 11A8 50-100 mesh). The synthesis was completed in 60 min following E.O.B. with an overall radiochemical yield of 12% (17.5% E.O.B.). See McConathy et al. (2003) supra for details.

### Example 2

### Synthesis of syn- and anti-1-amino-4-hydroxycyclohexane-1-carboxylic acid esters (Schemes 7-9)

4-Ethylene acetal cyclohexanol (16)

To a solution of 1,4-cyclohexanedione monoethylene acetal (3.41 g, 21.8 mmol) in 50 ml methanol cooled to 0° C. was added sodium borohydride (0.826 g, 21.8 mmol) in portions. The reaction was stirred for an additional 1.5 hr before being brought to pH 7 by the addition of 1 N HCl. The mixture was partitioned between ethyl acetate and brine. The aqueous layer was concentrated to the point that a precipitate began to form and this layer was extracted twice with ethyl acetate. The combined organic layers were dried over sodium sulfate, filtered and concentrated. This crude alcohol (3.28 g, 95.2%) was used without further purification. ¹H NMR (CDCl₃) δ: 1.54-1.87 (8H, m, 4×-CH₂—), 3.77 (1H, m, —CH—), 3.91 (4H, t, 2×O—CH₂—).

1-Ethylene acetal-4-benzyloxy-cyclohexane (17)

To a suspension of sodium hydride (410 mg, 17.1 mmol) in 15 ml THF at 0° C. was added 4-ethylene acetal cyclohexanol (1) (1.36 g, 8.61 mmol) in 5 ml THF. The reaction was stirred at 0° C. for 1.5 hr and benzyl bromide (1.75 g, 10.2 mmol) was added. The reaction was stirred at rt overnight. The reaction was quenched with ammonium chloride (sat.). The product was extracted with ethyl acetate and the organic phase was washed with brine, dried over sodium sulfate, filtered and concentrated. The crude product was purified by silica gel chromatography (20% ethyl acetate in hexane) to give 2.17 g (100%) of benzyl ether. ¹H NMR (CDCl₃) δ: 1.51-1.88 (8H, m, 4×-CH₂—, 3.51 (1H, m, —CH—), 3.91 (4H, t, 2×O—CH₂—), 4.52 (2H, s, Ph-CH₂—), 7.25-7.34 (5H, m, Ph-H).

4-Benzyloxy-cyclohexanone (18)

To a solution of 1-ethylene acetal-4-benzyloxy-cyclohexane (17) (3.13 g, 12.6 mmol) in 50 ml THF, aqueous hydrochloric acid (1N, 30 ml) was added at rt. The reaction was stirred overnight and neutralized with sodium bicarbonate (sat.). The product was extracted with ethyl acetate and the organic phase was washed with brine, dried over sodium sulfate, filtered and concentrated. Purification by the silica gel chromatography (20% ethyl acetate in hexane) yielded 2.45 g (95.2%) of the title ketone. ¹H NMR (CDCl₃) δ: 1.95-2.62 (8H, m, 4×-CH₂—), 3.82 (1H, m, —CH—), 4.59 (2H, s, Ph-CH₂—), 7.28-7.36 (5H, m, Ph-H).

Syn/anti-6-(4-benzyloxycyclohexane)hydantoins (19)

To a solution of 4-benzyloxy-cyclohexanone (18) (2.45 g, 12 mmol) in 100 ml of ethanol was added a solution of ammonium carbonate (4.6 g, 48 mmol) and ammonium chloride (1.28 g, 24 mmol) in 100 ml of water. The mixture was stirred at rt for 15 min and then potassium cyanide (940 mg, 14.4 mmol) was added. The reaction was stirred at rt overnight. The solvent was removed under reduced pressure. The resulting solid was washed repeatedly with water and collected by filtration. This crude syn/anti mixture of hytantoins (3.02 g, 91.8%) was used without further purification. ¹H NMR (CD₃OD) δ: 1.58-2.15 (8H, m, 4×-CH₂—), 3.48, 3.66 (1H, m, —CH—), 4.52, 4.56 (2H, s, Ph-CH₂—), 7.25-7.33 (5H, m, Ph-H).

Syn/anti-1-amino-4-benzyloxyycclohexane-1-carboxylic acids (20)

The syn/anti hytantoins (19) (2.72 g, 9.93 mmol) were suspended in 30 ml 3N NaOH and sealed in a steel cylinder which was heated at 120° C. for 1 day. After cooling to rt, the reaction was brought to pH 7 by addition of concentrated hydrochloric acid solution. The crude product of syn/anti amino acids was obtained by concentrating to dryness under reduced pressure. This product was used without further purification.

Syn/anti-1-[N-(t-butoxycarbonyl)amino]-4-benzyloxycyclohexane-1-carboxylic acids (21)

To a suspension of syn/anti-1-amino-4-benzyloxycyclohexane-1-carboxylic acids (20) from above preparation in 50 ml 9:1 MeOH/triethylamine was added di-t-butyl dicarbonate (3.25 g, 14.9 mmol). The reaction mixture was stirred at rt for 24 hrs. The solvent was removed under reduced pressure. The resulting residue was dissolved in 50 ml of ice cold 1:1 water/ethyl acetate. The pH of the solution was adjusted to 2-3 with 3N HCl. The organic layer was retained while the aqueous layer was saturated with sodium chloride and extracted with ethyl acetate (3×25 ml). The combined organic layers were dried over magnesium sulfate and the solvent was removed under reduced pressure. This product (3.46 g, 100%) was used without further purification. ¹H NMR (CD₃OD) δ: 1.41 [9H, s, —C(CH₃)₃], 1.57-2.25 (8H, m, 4×-CH₂—), 3.40, 3.58 (1H, m, —CH—), 4.49, 4.54 (2H, s, Ph-CH₂—), 4.84 (1H, br, NH), 7.25-7.33 (5H, m, Ph-H).

Syn/anti-1-[N-(t-butoxycarbonyl)amino]-4-benzyloxycyclohexane-1-carboxylic acid methyl esters (22)

Syn/anti-1-[N-(t-butoxycarbonyl)amino]-4-benzyloxycyclohexane-1-carboxylic acids (21) (1.14 g, 3.26 mmol) were dissolved in 40 ml benzene and 10 ml methanol and trimethylsilyl diazomethane (558 mg, 4.88 mmol, 2.5 ml of 2M solution in hexane) was added at rt. The reaction was stirred at rt for 30 min then the solvent was removed under reduced pressure. Purification by flush chromatography with 20% ethyl acetate in hexane afforded 1.03 g (87.2%) of pure product as an oil. ¹H NMR (CD₃OD) δ: 1.408, 1.413 [9H, s, —C(CH₃)₃], 1.5-2.3 (8H, m, 4×-CH₂—), 3.40, 3.58 (1H, m, —CH—), 3.69, 3.71 (3H, s, COCH₃), 4.49, 4.54 (2H, s, Ph-CH₂—), 4.77, 4.79 (1H, br, NH), 7.25-7.33 (5H, m, Ph-H).

Syn/anti-1-[N-(t-butoxycarbonyl)amino]-4-hydroxycyclohexane-1-carboxylic acid methyl esters (23)

A suspension of the benzyl ethers (22) (947 mg, 2.6 mmol) and 10% palladium on charcoal (142 mg) in 50 ml of ethanol was stirred under a hydrogen atmosphere overnight. The reaction mixture was filtered over Celite®, and the filtrate was concentrated under reduced pressure. Purification via silica gel column chromatography (50% ethyl acetate in hexane) provided a yield of (23) (701 mg, 98.4%), anti- to syn-ratio was 34:66. ¹H NMR (CD₃OD) δ: 1.411, 1.416 [9H, s, —C(CH₃)₃], 1.53-2.25 (8H, m, 4×-CH₂—), 3.65, 3.91 (1H, m, —CH—), 3.70, 3.71 (3H, s, COCH₃), 4.77 (1H, br, NH).

1-[N-(t-Butoxycarbonyl)amino]-4-cyclohexanone-1-carboxylic acid methyl ester (24)

Tetrapropyl ammonium perruthenate (26 mg, 0.075 mmol) was added in one portion to a stirring mixture of alcohols (23) (410 mg, 1.5 mmol), N-methyl-morpholine N-oxide (264 mg, 2.25 mmol) and 750 mg 4A molecular sieves in 15 ml of 10% acetonitrile in dichloromethane under argon. The reaction was stirred at rt for 1 hr then the solvent was removed under reduced pressure. The resulting residue was taken into dichloromethane and purified with silica gel column chromatography (30% ethyl acetate in hexane). The ketone (24), 372 mg (91.6%), was obtained as a white solid. ¹H NMR (CD₃OD) δ: 1.43 [9H, s, —C(CH₃)₃], 2.32-2.42 (8H, m, 4×-CH₂—), 3.74 (3H, s, COCH₃), 5.04 (1H, br, NH).

1-Amino-4-cyclohexanon-1-carboxylic acid methyl ester (25)

To a solution of the ketone (24) (325 mg, 1.2 mmol) in 5 ml dichloromethane was added trifluoroacetic acid (1.37 g, 12 mmol). The reaction was stirred at rt overnight. The solvent and reagent were removed under reduced pressure. The resulting white solid was used without further purification.

1-[N-(Phthaloyl)amino]-4-cyclohexa non-1-carboxylic acid methyl ester (26b)

To the suspension of the amine (25) (80 mg, 0.47 mmol) and triethylamine (476 mg, 4.7 mmol) in 10 ml toluene was added phthalic anhydride (77 mg, 0.52 mmol). The mixture was refluxed at 120° C. for 5 hrs. The reaction was washed with brine and aqueous layer was extracted with ethyl acetate. The combined organic layers were dried over sodium sulfate, filtered and concentrated. The crude product was purified by flush chromatography with 1:4 ethyl acetate and hexane to give the ketone (26b) (37.6 mg, 26.6%, 2 steps) as a white solid. ¹H NMR (CD₃OD) δ: 2.54-3.14 (8H, m, 4×-CH₂—), 3.77 (3H, s, COCH₃), 7.73-7.85 (4H, m, Ph-H).

1-[N-(Trifluoroacetyl)amino]-4-cyclohexanon-1-carboxylic acid methyl ester (26c)

To the suspension of the amine (25) (14 mg, 0.082 mmol) and triethylamine (166 mg, 1.64 mmol) in 1 ml dichloromethane cooled to −10° C. was added trifluoroacetic anhydride (86 mg, 0.41 mmol). The mixture was warmed to rt and stirred overnight. A few drops of 1 N ammonium chloride was added and stirred for 30 min. The reaction was washed with brine and aqueous layer was extracted with ethyl acetate. The combined organic layers were dried over sodium sulfate, filtered and concentrated. The crude product was purified by flush chromatography with 1:2 ethyl acetate and hexane to give the ketone (26c) (17.5 mg, 79.9%) as clear oil. ¹H NMR (CD₃OD) δ: 2.44-2.56 (8H, m, 4×-CH₂—), 3.79 (3H, s, COCH₃), 6.86 (1H, br, NH).

1-[N-(Benzoyl)amino]-4-cyclohexanon-1-carboxylic acid methyl ester (26d)

To the suspension of the amine (25) (50 mg, 0.29 mmol) and pyridine (934 mg, 11.8 mmol) in 3 ml dichloromethane cooled to 0° C. was added benzoyl chloride (62 mg, 0.44 mmol). The mixture was warmed to rt and stirred overnight. The reaction was washed with brine and aqueous layer was extracted with ethyl acetate. The combined organic layers were dried over sodium sulfate, filtered and concentrated. The crude product was purified by flush chromatography with 1:2 ethyl acetate and hexane to give the ketone (26d) (22 mg, 27.6%) as a white solid. ¹H NMR (CD₃OD) δ: 2.46-2.58 (8H, m, 4×-CH₂—), 3.81 (3H, s, COCH₃), 6.82 (1H, br, NH), 7.48-8.13 (5H, m, Ph-H).

Syn/anti-1-[N-(t-butoxycarbonyl)amino]-4-hydroxycyclohexane-1-carboxylic acid methyl esters (27a)

To the solution of the ketone (26a) (18 mg, 0.066 mmol) in 1 ml THF was added zinc chloride (18 mg, 0.13 mmol, 264 μl of 0.5 M solution in THF) at rt and the mixture was stirred for 30 min. The reaction was cooled to −78° C. and L-selectride (19 mg, 0.10 mmol, 100 μl of 1 M solution in THF) was added. The mixture was stirred at −78° C. for 2 hrs and at rt overnight. A few drops of 1 N ammonium chloride was added and stirred for 30 min. The reaction was washed with brine and aqueous layer was extracted with ethyl acetate. The combined organic layers were dried over sodium sulfate, filtered and concentrated. The crude product was purified by flush chromatography with 1:1 ethyl acetate and hexane to give the alcohols (27a) (12.7 mg, 70.5%) as clear oil, anti- to syn- ratio was 67:33. ¹H NMR (CD₃OD) δ: 1.411, 1.415 [9H, s, —C(CH₃)₃], 1.55-2.26 (8H, m, 4×-CH₂—), 3.65, 3.92 (1H, m, —CH—), 3.70, 3.71 (3H, s, COCH₃), 4.70 (1H, br, NH).

Syn/anti-1-[N-(t-butoxycarbonyl)amino]-4-hydroxycyclohexane-1-carboxylic acid methyl esters (27a) (absence of zinc chloride)

To the solution of the ketone (24) (21.7 mg, 0.08 mmol) in 1 ml THF cooled to −78° C. was added L-selectride (22.8 mg, 0.12 mmol, 120 μl of 1 M solution in THF). The mixture was stirred at −78° C. for 2 hrs and at rt overnight. A few drops of 1 N ammonium chloride was added and stirred for 30 min. The reaction was washed with brine and aqueous layer was extracted with ethyl acetate. The combined organic layers were dried over sodium sulfate, filtered and concentrated. The crude product was purified by flush chromatography with 1:1 ethyl acetate and hexane to give the alcohols (27a) (3 mg, 13.7%) as clear oil, anti- to syn-ratio was 11:89. ¹H NMR (CD₃OD) δ: 1.415, 1.420 [9H, s, —C(CH₃)₃], 1.53-2.25 (8H, m, 4×-CH₂), 3.65 (1H, m, —CH—), 3.70, 3.71 (3H, s, COCH₃), 4.70 (1H, br, NH).

Syn/anti-1-[N-(phthaloyl)amino]-4-hydroxycyclohexane-1-carboxylic acid methyl esters (27b)

To the solution of the ketone (26b) (20 mg, 0.066 mmol) in 1 ml THF was added zinc chloride (18 mg, 0.13 mmol, 260 μl of 0.5 M solution in THF) at rt and the mixture was stirred for 30 min. The reaction was cooled to −78° C. and L-selectride (19 mg, 0.10 mmol, 100 μl of 1 M solution in THF) was added. The mixture was stirred at −78° C. for 2 hrs and at rt overnight. A few drops of 1 N ammonium chloride was added and stirred for 30 min. The reaction was washed with brine and aqueous layer was extracted with ethyl acetate. The combined organic layers were dried over sodium sulfate, filtered and concentrated. The crude product was purified by flush chromatography with 1:1 ethyl acetate and hexane to give the alcohols (27b) (13.2 mg, 66%) as clear oil, anti- to syn-ratio was 52:48. ¹H NMR (CD₃OD) δ: 1.60-2.01 (8H, m, 4×-CH₂), 3.70, 3.75 (3H, s, COCH₃), 3.86 (1H, m, —CH—), 7.69-7.82 (4H, m, Ph-H).

Syn/anti-1-[N-(phthaloyl)amino]-4-hydroxycyclohexane-1-carboxylic acid methyl esters (27b) (absence of zinc chloride)

To the solution of the ketone (26b) (18 mg, 0.059 mmol) in 1 ml THF cooled to −78° C. was added L-selectride (17 mg, 0.09 mmol, 90 μl of 1 M solution in THF). The mixture was stirred at −78° C. for 2 hrs and at rt overnight. A few drops of 1 N ammonium chloride was added and stirred for 30 min. The reaction was washed with brine and aqueous layer was extracted with ethyl acetate. The combined organic layers were dried over sodium sulfate, filtered and concentrated. The crude product was purified by flush chromatography with 1:1 ethyl acetate and hexane to give the alcohols (27b) (13.2 mg, 66%) as clear oil, anti- to syn-ratio was 52:48.

Syn/anti-1-[N-(trifuoroacetyl)amino]-4-hydroxycyclohexane-1-carboxylic acid methyl esters (27c)

To the solution of the ketone (26c) (17 mg, 0.064 mmol) in 1 ml THF was added zinc chloride (17 mg, 0.13 mmol, 256 μl of 0.5 M solution in THF) at rt and the mixture was stirred for 30 min. The reaction was cooled to −78° C. and L-selectride (18 mg, 0.096 mmol, 96 μl of 1 M solution in THF) was added. The mixture was stirred at −78° C. for 2 hrs and at rt overnight. A few drops of 1 N ammonium chloride was added and stirred for 30 min. The reaction was washed with brine and aqueous layer was extracted with ethyl acetate. The combined organic layers were dried over sodium sulfate, filtered and concentrated. The crude product was purified by flush chromatography with 1:1 ethyl acetate and hexane to give the alcohols (27c) (13.5 mg, 78.4%) as clear oil, anti- to syn-ratio was 66:34. ¹H NMR (CD₃OD) δ: 1.67-2.37 (8H, m, 4×-CH₂—), 3.72, 3.75 (3H, s, COCH₃), 3.97 (1H, m, —CH—), 6.43 (1H, br, NH).

Syn/anti-1-[N-(benzoyl)amino]-4-hydroxycyclohexane-1-carboxylic acid methyl esters (27d)

To the solution of the ketone (26d) (22 mg, 0.08 mmol) in 1 ml THF was added zinc chloride (22 mg, 0.16 mmol, 320 μl of 0.5 M solution in THF) at rt and the mixture was stirred for 30 min. The reaction was cooled to −78° C. and L-selectride (23 mg, 0.12 mmol, 120 μl of 1 M solution in THF) was added. The mixture was stirred at −78° C. for 2 hrs and at rt overnight. A few drops of 1 N ammonium chloride was added and stirred for 30 min. The reaction was washed with brine and aqueous layer was extracted with ethyl acetate. The combined organic layers were dried over sodium sulfate, filtered and concentrated. (The product could not be detected).

### Example 3

### Amino Acid Uptake Assays in Vitro and in Vivo

The tumor cells were initially grown as monolayers in T-flasks containing Dulbecco's Modified Eagle's Medium (DMEM) under humidified incubator conditions (37° C., 5% CO₂/95% air). The growth media were supplemented with 10% fetal calf serum and antibiotics (10,000 units/ml penicillin and 10 mg/ml streptomycin). The growth media were replaced three times per week, and the cells were passaged so the cells would reach confluency in a week's time.

When the monolayers were confluent, cells were prepared for experimentation in the following manner. Growth media were removed from the T-flask, and the monolayer cells were exposed to 1× trypsin:EDTA for ˜1 minute to weaken the protein attachments between the cells and the flask. The flask was then slapped, causing the cells to release. Supplemented media were added to inhibit the proteolytic action of the trypsin, and the cells were aspirated through an 18 Ga needle until they were monodispersed. A sample of the cells was counted under a microscope using a hemocytometer, and the live/dead fraction estimated through trypan blue staining (>98% viability). The remainder of the cells was placed into a centrifuge tube, centrifuged at 75×g for 5 minutes, and the supernatant was removed. The cells were then resuspended in amino-acid/serum-free DMEM salts.

In this study, approximately 4.55×10⁵ cells were exposed to either [¹⁸F]10 (anti-FACBC) or [¹⁸F]15 (syn-FACBC, 5 μCi) in 3 ml of amino acid free media ±transporter inhibitors (10 mM) for 30 minutes under incubator conditions in 12×75 mm glass vials. Each assay condition was performed in duplicate. After incubation, cells were twice centrifuged (75×g for 5 minutes) and rinsed with ice-cold amino-acid/serum-free DMEM salts to remove residual activity in the supernatant. The vials were placed in a Packard Cobra II Auto-Gamma counter, the raw counts decay corrected, and the activity per cell number determined. The data from these studies (expressed as percent uptake relative to control) were graphed using Excel, with statistical comparisons between the groups analyzed using a 1-way ANOVA (GraphPad Prism software package).

To test the hypothesis that [¹⁸F]10 and [¹⁸F]15 enter cells predominantly via the L-type amino acid transport system, amino acid uptake assays using cultured 9L gliosarcoma and a variety of human cancer cell lines in the presence and absence of two well-described inhibitors of amino acid transport were performed. N-MeAIB is a selective competitive inhibitor of the A-type amino acid transport system while 2-amino-bicyclo[2.2.1]heptane-2-carboxylic acid (BCH) is commonly used as an inhibitor for the sodium-independent L-type transport system, although this compound also competitively inhibits amino acid uptake via the sodium-dependent B^{0,+} and B⁰ transport systems. The A- and L-type amino acid transport systems have been implicated in the in vivo uptake of radiolabeled amino acids used for tumor imaging.

In the absence of inhibitors, both [¹⁸F]10 and [¹⁸F]15 showed similar levels of uptake in 9L gliosarcoma cells and a variety of human cancer cell lines, with intracellular accumulations of 0.43% and 0.50% of the initial dose per million cells after 30 minutes of incubation, respectively. To facilitate the comparison of the effects of the inhibitors, the data were expressed as percent uptake relative to the control condition (no inhibitor) as shown in Table 1. In the case of [¹⁸F]10 and [¹⁸F]15, BCH blocked >50% of the uptake of activity relative to controls. The reduction of uptake of [¹⁸F]10 and [¹⁸F]15 by BCH compared to controls was statistically significant (p<0.05, p<0.01 respectively by 1-way ANOVA). These inhibition studies indicate that [¹⁸F]10 and [¹⁸F]15 are substrates for the L-type amino acid transport system in the cancer cells studied based on the inhibition of uptake of both compounds in the presence of BCH.

TABLE 1
Uptake of syn- and anti-[¹⁸F]FACBC in tumor cells expressed
as percent uptake relative to control.

| | | DU145 | SKOV3 | U87 | A549 | MB 468 |
|---|---|---|---|---|---|---|
| | | Prostate | Ovarian | Glioma | Lung | Breast |
| Syn-[¹⁸F]FACBC | No | 20.27 | 11.67 | 24.77 | 11.91 | 33.53 |
| | inhib- | | | | | |
| | itor | | | | | |
| | BCH | 9.11 | 5.87 | 5.00 | 3.85 | 10.93 |
| | MeAIB | 17.16 | 8.48 | 14.80 | 9.61 | 28.25 |
| Anti-[¹⁸F]FACBC | No | 16.06 | 4.68 | 3.41 | 12.17 | 15.51 |
| | inhib- | | | | | |
| | itor | | | | | |
| | BCH | 4.16 | 1.44 | 1.51 | 2.69 | 4.43 |
| | MeAIB | 13.90 | 6.39 | 3.45 | 11.02 | 14.80 |

Tumor Induction and Animal Preparation:

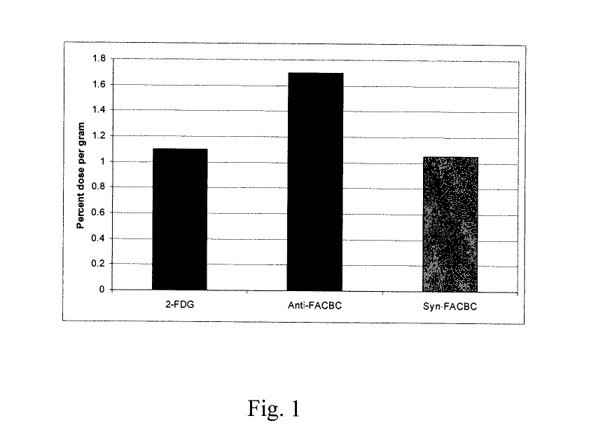

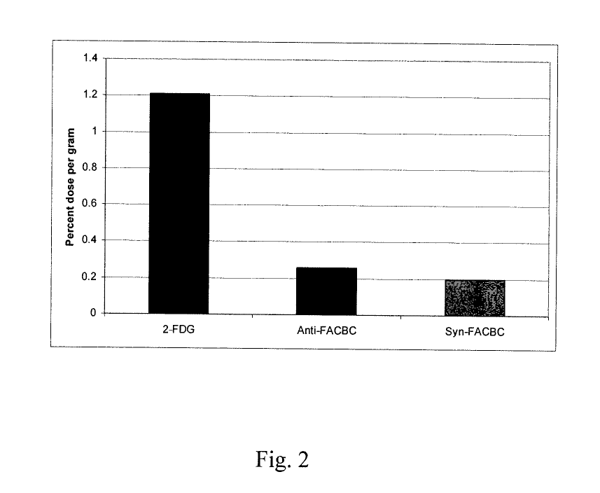

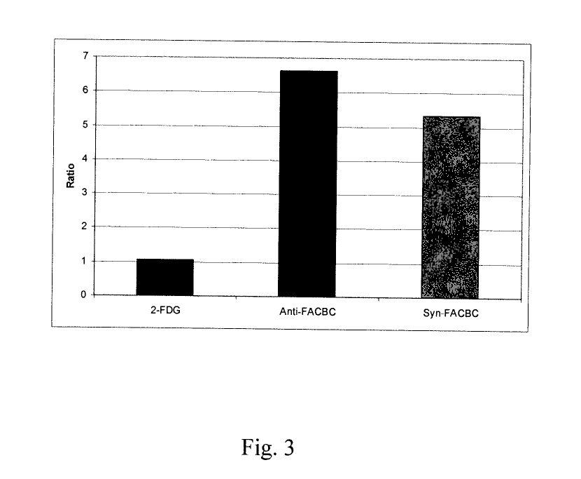

All animal experiments were carried out under humane conditions and were approved by the Institutional Animal Use and Care Committee (IUCAC) at Emory University. Rat 9L gliosarcoma cells were implanted into the brains of male Fischer rats. Briefly, anesthetized rats placed in a stereotactic head holder were injected with a suspension of 4×10⁴ rat 9L gliosarcoma cells (1×10⁷ per mL) in a location 3 mm right of midline and 1 mm anterior to the bregma at a depth of 5 mm deep to the outer table. The injection was performed over the course of 2 minutes, and the needle was withdrawn over the course of 1 minute to minimize the backflow of tumor cells. The burr hole and scalp incision were closed, and the animals were returned to their original colony after recovering from the procedure. Intracranial tumors developed that produced weight loss, apathy and hunched posture in the tumor-bearing rats, and the animals were used at 17-19 days after implantation. Of the 30 animals implanted with tumor cells, 25 developed tumors visible to the naked eye upon dissection and were used in the study. FIGS. 1-3 show the results of these studies.

Rodent Biodistribution Studies:

The tissue distribution of radioactivity was determined in 16 normal male Fischer 344 rats (200-250 g) after intravenous injection of ˜85 μCi of [¹⁸F]10 or [¹⁸F]15 in 0.3 mL of sterile water. The animals were allowed food and water ad libitum before the experiment. The tail vein injections were performed in awake animals using a RTV-190 rodent restraint device (Braintree Scientific) to avoid mortality accompanying anesthesia in the presence of an intracranial mass. Groups of four rats were killed at 5 minutes, 30 minutes, 60 minutes and 120 minutes after injection of the dose. The animals were dissected, and selected tissues were weighed and counted along with dose standards in a Packard Cobra II Auto-Gamma Counter. The raw counts were decay corrected, and the counts were normalized as the percent of total injected dose per gram of tissue (% ID/g). A comparison of the uptake of activity in tumor tissue, and the corresponding region of brain contralateral to the tumor was excised and used for comparison. at each time point was analyzed using a 1-way ANOVA (GraphPad Prism software package). FIGS. 1-3 below show the results of these studies.

As seen in FIGS. 1-3, in rats implanted intracranially with 9L gliosarcoma cells, the retention of radioactivity in tumor tissue was high at 60 minutes after intravenous injection of [¹⁸F]10 and [¹⁸F]15 while the uptake of radioactivity in brain tissue contralateral to the tumor remained low (<0.3% dose/g). Ratios of tumor uptake to normal brain uptake for [¹⁸F]10 was 6.5:1 at 60 and 120 minutes, while for [¹⁸F]15 the ratios was 5.3:1 at the same time point. These results demonstrate that like anti-[¹⁸F]FACBC, [¹⁸F]10, syn-[¹⁸F]FACBC [¹⁸F]15 is an excellent candidate for imaging brain tumors.

The compounds made by the inventive method may also be solvated, especially hydrated. Hydration may occur during manufacturing of the compounds or compositions comprising the compounds, or the hydration may occur over time due to the hygroscopic nature of the compounds. In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

When the compounds of the invention are to be used as imaging agents, they must be labeled with suitable radioactive halogen isotopes such as ¹²³I, ¹³¹I, ¹⁸F, ⁷⁶Br, and ⁷⁷Br. The radiohalogenated compounds of this invention can easily be provided in kits with materials necessary for imaging a tumor. For example, a kit can contain a final product labeled with an appropriate isotope (e.g. ¹⁸F) ready to use for imaging or an intermediate compound and a label (e.g. K[¹⁸F]F) with reagents (e.g. solvent, deprotecting agent) such that a final product can be made at the site or time of use.

In the first step of the method of tumor imaging, a labeled compound of the invention is introduced into a tissue or a patient in a detectable quantity. The compound is typically part of a pharmaceutical composition and is administered to the tissue or the patient by methods well known to those skilled in the art. For example, the compound can be administered either orally, rectally, parenterally (intravenous, by intramuscularly or subcutaneously), intracistemally, intravaginally, intraperitoneally, intravesically, locally (powders, ointments or drops), or as a buccal or nasal spray.

In an imaging method of the invention, the labeled compound is introduced into a patient in a detectable quantity and after sufficient time has passed for the compound to become associated with tumor tissues or cells, the labeled compound is detected noninvasively inside the patient. In another embodiment of the invention, a labeled compound is introduced into a patient, sufficient time is allowed for the compound to become associated with tumor tissues, and then a sample of tissue from the patient is removed and the labeled compound in the tissue is detected apart from the patient. Alternatively, a tissue sample is removed from a patient and a labeled compound of the invention is introduced into the tissue sample. After a sufficient amount of time for the compound to become bound to tumor tissues, the compound is detected. The term “tissue” means a part of a patient's body. Examples of tissues include the brain, heart, liver, blood vessels, and arteries. A detectable quantity is a quantity of labeled compound necessary to be detected by the detection method chosen. The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The administration of the labeled compound to a patient can be by a general or local administration route. For example, the labeled compound may be administered to the patient such that it is delivered throughout the body. Alternatively, the labeled compound can be administered to a specific organ or tissue of interest.

Those skilled in the art are familiar with determining the amount of time sufficient for a compound to become associated with a tumor. The amount of time necessary can easily be determined by introducing a detectable amount of a labeled compound of the invention into a patient and then detecting the labeled compound at various times after administration.

Those skilled in the art are familiar with the various ways to detect labeled compounds. For example, magnetic resonance imaging (MRI), positron emission tomography (PET), or single photon emission computed tomography (SPECT) can be used to detect radiolabeled compounds. PET and SPECT are preferred when the compounds of the invention are used as tumor imaging agents. The label that is introduced into the compound will depend on the detection method desired. For example, if PET is selected as a detection method, the compound must possess a positron-emitting atom, such as ¹¹C or ¹⁸F.

The radioactive diagnostic agent should have sufficient radioactivity and radioactivity concentration which can assure reliable diagnosis. For instance, in case of the radioactive metal being technetium-99m, it may be included usually in an amount of 0.1 to 50 mCi in about 0.5 to 5.0 ml at the time of administration. The amount of a compound of formula may be such as sufficient to form a stable chelate compound with the radioactive metal.

The inventive compound as a radioactive diagnostic agent is sufficiently stable, and therefore it may be immediately administered as such or stored until its use. When desired, the radioactive diagnostic agent may contain any additive such as pH controlling agents (e.g., acids, bases, buffers), stabilizers (e.g., ascorbic acid) or isotonizing agents (e.g., sodium chloride). The imaging of a tumor can also be carried out quantitatively using the compounds herein so that a therapeutic agent for a given tumor can be evaluated for its efficacy.

Preferred compounds for imaging include a radioisotope such as ¹²³I, ¹²⁴I, ¹²⁵I, ¹³¹I, ¹⁸F, ⁷⁶Br, ⁷⁷Br or ¹¹C.

The synthetic schemes described herein represent exemplary syntheses of preferred embodiments of the present invention. However, one of ordinary skill in the art will appreciate that starting materials, reagents, solvents, temperature, solid substrates, synthetic methods, purification methods, analytical methods, and other reaction conditions other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

When a group of substituents is disclosed herein, it is understood that all individual members of that group and all subgroups, including any isomers and enantiomers of the group members, are disclosed separately. When a Markush group or other grouping is used herein, all individual members of the group and all combinations and subcombinations possible of the group are intended to be individually included in the disclosure. When a compound is described herein such that a particular isomer or enantiomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions, those that are appropriate for preparation of salts of this invention for a given application.

Every formulation or combination of components described or exemplified herein can be used to practice the invention, unless otherwise stated.

Whenever a range is given in the specification, for example, a temperature range, a time range, a purity range or a composition or concentration range, all intermediate ranges and subranges, as well as all individual values included in the ranges given are intended to be included in the disclosure.

All patents and publications mentioned in the specification are indicative of the levels of skill of those in the art to which the invention pertains. References cited herein are incorporated by reference herein in their entirity to indicate the state of the art as of their filing date and it is intended that this information can be employed herein, if needed, to exclude specific embodiments that are in the prior art. For example, when a compound is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

All references cited herein are hereby incorporated by reference to the extent that there is no inconsistency with the disclosure of this specification. In particular, U.S. Pat. Nos. 5,808,146, 5,817,776, and WO 03/093412 are cited herein and incorporated by reference herein to provide examples of the amino cid analogs that can be made using the invention and the detailed synthetic methods. Some references provided herein are incorporated by reference to provide details concerning sources of starting materials, additional starting materials, additional reagents, additional methods of synthesis, additional methods of analysis and additional uses of the invention.

We claim:

1. A method of synthesizing a substantially pure syn-amino acid analog of formula II, wherein formula II is 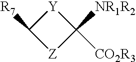
wherein Y & Z are independently selected from the group consisting of CH₂, N, O, S, Se, and (CR₄, R₅)n, n=1-4; R₁-R₃ are independently selected from the group consisting of H, alkyl, cycloalkyl, acyl, aryl, alkenyl, alkynyl, haloalkyl, haloacyl, heteroaryl, haloaryl, haloheteroaryl, haloalkenyl, and haloalkynyl; R₄-R₅ are independently selected from the group consisting of H, alkyl, cycloalkyl, acyl, aryl, halo, haloalkyl, haloacyl, heteroaryl, haloaryl, haloheteroaryl, alkenyl, alkynyl, haloalkenyl, and haloalkynyl, where halo is selected from the group consisting of non-radioactive F, Cl, Br, and I; R7 is selected from the group consisting of halogen, haloalkyl, haloalkenyl, haloalkynyl, haloheteroalkyl, haloheteroalkenyl, haloheteroalkynyl, haloaryl, and haloheteroaryl, Tc-99m and Re chelates thereof, where halo or halogen is selected from the group consisting of F, Cl, Br, I, At, F-18, I-123, I-124 and Br-76; or a pharmaceutically acceptable salt thereof, comprising steps of converting a ketone to a trans-alcohol of formula I, and converting the trans-alcohol to the syn-amino acid analog of formula II, wherein formula I is 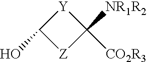
wherein Y & Z are independently selected from the group consisting of CH₂, N, O, S, Se and (CR₄, R₅)n, n=1-4; R₁-R₃ are independently selected from the group consisting of H, alkyl, cycloalkyl, acyl, aryl, alkenyl, alkynyl, haloalkyl, haloacyl, heteroaryl, haloaryl, haloheteroaryl, haloalkenyl, and haloalkynyl; R₄ and R₅ are independently selected from the group consisting of H, alkyl, cycloalkyl, acyl, aryl, halo, haloalkyl, haloacyl, heteroaryl, haloaryl, haloheteroaryl, alkynyl, alkenyl, haloalkenyl, and haloalkynyl, where halo is selected from the group consisting of non-radioactive F, Cl, Br, and I.

2. The method of claim 1 wherein R₄ and R₅ are selected independently from the group consisting of H, alkyl, cycloalkyl, acyl, aryl, heteroaryl, alkynyl, and alkenyl; R₇ is selected from the group consisting of halogen, haloalkyl_{C1-C6}, haloalkenyl_{C1-C6}, haloalkynyl_{C1-C6}, haloheteroalkyl, haloheteroalkenyl, haloheteroalkynyl, haloaryl, and haloheteroaryl, where halo or halogen in R₇ is either ¹⁸F or ¹²³I.

3. The method of claim 2 wherein R₁, R₂, and R₃ are selected independently from the group consisting of hydrogen, alkyl_{C1-C6}, haloalkyl_{C1-C6}, alkenyl_{C1-C}₆, haloalkenyl_{C1-C6}, alkynyl_{C1-C6}, and haloalkynyl_{C1-C6}

4. The method of claim 3 wherein Y and Z in the amino acid analog are CH₂.

5. The method of claim 4 wherein R₁, R₂, and R₃ are hydrogen or alkyl_{C1-C4}.

6. The method of claim 1 or 5 wherein R₇ is selected from the group consisting of ¹⁸F, ¹⁸F-alkyl_{C1-C4}, ¹²³I and ¹²³I-alkyl_{C1-C4}.

7. The method of claim 6 wherein the amino acid analog is syn-3-[¹⁸F]FACBC.

8. The method of claim 6 wherein the amino acid analog is syn-3-[¹²³I]IACBC.

9. The method of claim 6 wherein the amino acid analog is syn-3-[¹⁸F]FMACBC.

10. The method of claim 6 wherein the amino acid analog is syn-[¹⁸F]FACHC.

11. A substantially pure compound of the formula: 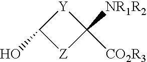
wherein Y & Z are independently selected from the group consisting of CH₂, N, O, S, Se and (CR₄, R₅)n, n=1-4; R₁-R₃ are independently selected from the group consisting of H, alkyl, cycloalkyl, acyl, aryl, alkenyl, alkynyl, haloalkyl, haloacyl, heteroaryl, haloaryl, haloheteroaryl, haloalkenyl, and haloalkynyl; R₄-R₅ are independently selected from the group consisting of H, alkyl, cycloalkyl, acyl, aryl, halo, haloalkyl, haloacyl, heteroaryl, haloaryl, haloheteroaryl, alkynyl, alkenyl, haloalkenyl, and haloalkynyl, where halo is selected from the group consisting of non-radioactive F, Cl, Br, and I.

12. The compound of claim 11 wherein R₁, R₂, and R₃ are selected independently from the group consisting of H, alkyl_{C1-C6}, haloalkyl_{C1-C6}, alkenyl_{C1-C6}, alkynyl_{C1-C6}, haloalkenyl_{C1-C6} and haloalkynyl_{C1-C6}; R₄ and R₅ are selected independently from the group consisting of hydrogen, alkyl_{C1-C6}, aryl, heteroaryl, alkynyl_{C1-C6}, and alkenyl_{C1-C6}.

13. The compound of claim 12 wherein R₁, R₂, and R₃ are hydrogen, and Y and Z are CH₂.

14. The compound of claim 12 wherein R₁, R₂, and R₃ are hydrogen, and Y and Z are C₂H₄.

15. A substantially pure syn-amino acid analog made by the method of claim 1.

16. The amino acid analog of claim 15 wherein the analog is syn-3-[¹⁸F]FACBC.

17. A pharmaceutical composition for imaging a tumor, comprising the syn-amino acid analog of claim 15 and a physiologically acceptable carrier.

18. The composition of claim 17 wherein the amino acid analog is syn-3-[¹⁸F]FACBC.

19. A method of tumor imaging by positron emission tomography or single photon emission computed tomography, comprising: a) administering to a subject suspected of having a tumor an image-generating amount of a labeled compound of claim 1; b) allowing sufficient time for the labeled compound to become associated with the tumor; and c) measuring the distribution of the labeled compound in the subject by PET or SPECT.

20. The method of claim 19 wherein the labeled compound is syn-3-[¹⁸F]FACBC.

21. A kit for synthesizing a substantially pure syn-3-[¹⁸F]FACBC comprising the compound of claim 11 and reagents necessary for converting the compound to syn-3-[¹⁸F]FACBC.
